(19) United States
(12) Patent Application Publication
(10) Pub. No.: US 2014/0365137 A1
(43) Pub. Date: Dec. 11, 2014

(54) SYSTEM, METHOD AND COMPUTER-ACCESSIBLE MEDIUM FOR QUANTIFYING FDG UPTAKE ON PET

(71) Applicant: MEMORIAL SLOAN-KETTERING CANCER CENTER, New York, NY (US)

(72) Inventors: Irene A. Burger, Utikon Waldegg (CH); Charles Ross Schmidtlein, New York, NY (US)

(73) Assignee: MEMORIAL SLOAN-KETTERING CANCER CENTER, New York, NY (US)

(21) Appl. No.: 14/297,853

(22) Filed: Jun. 6, 2014

Related U.S. Application Data
(60) Provisional application No. 61/831,801, filed on Jun. 6, 2013.

(51) Int. Cl. A61B 5/145; G06F 17/18

(52) U.S. Cl. CPC A61B 5/14532; G06F 17/18
USPC 702/19

(57) ABSTRACT

The increasing use of molecular imaging probes as biomarkers in oncology can emphasize the need for robust and stable methods for quantifying tracer uptake on PET. A histogram-based system, method and computer-accessible procedure can be used to calculate a new tracer uptake metric and the background subtracted lesion activity.

### CROSS-REFERENCE TO RELATED APPLICATIONS

This application relates to and claims priority from U.S. Patent Application No. 61/831,801, filed on Jun. 6, 2013, the entire disclosure of which is incorporated herein by reference.

### FIELD OF THE DISCLOSURE

The present disclosure relates generally to quantifying [¹⁸F]-Fluorodeoxyglucose (“FDG”) uptake on positron emission tomography (“PET”), and more specifically, to exemplary embodiments of an exemplary system, method and computer-accessible medium for quantifying FDG uptake on PET using, for example, background subtracted lesion activity (“BSL”).

### BACKGROUND INFORMATION

Increasing use of molecular imaging probes as biomarkers in oncologic disease can emphasize the demand for robust and stable procedures to quantify radiotracer uptake on PET. (See, e.g., References 1-3). A commonly used procedure to quantify FDG uptake on PET can be the maximum standard uptake value (“SUVₘₐₓ”). (See, e.g., Reference 4). The ease of use and the excellent inter-observer reproducibility in combination with promising results for SUV as a prognostic factor, has led to its wide acceptance and routine clinical use. (See, e.g., Reference 5). However, there can be many disadvantages to the use of SUVₘₐₓ, particularly regarding the high statistical noise associated with a single voxel analysis. (See, e.g., References 6-8). Alternative quantitative metrics that take into account not just the SUVₘₐₓ but also the tracer uptake of the entire lesion have been proposed. One exemplary metric can be the total lesion glycolysis (“TLG”). (See, e.g., Reference 9). The TLG can be calculated by multiplying the total number of voxels within a volume of interest (“VOI”) that have an uptake above a predetermined SUV threshold by the mean SUV of all the voxels in the same VOI. Different SUV thresholds have been suggested. Two commonly used procedures include all voxels above 42% of the SUVmax TLG_{42%} or all voxels with an SUV over 2.5 TLG_{2.5}. (See, e.g., References 1 and 10-12). Increasing enthusiasm for the use of TLG can be evidenced through multiple reports describing its superiority over SUVmax as a predictive and prognostic biomarker in multiple tumors of the head and neck (see, e.g., Reference 13), gynecological organs (see, e.g., References 12 and 14), lung (see, e.g., References 15 and 16) and esophagus. (See, e.g., Reference 17). A PubMed search reveals that 34 of the total 59 papers analyzing TLG in FDG PET were published between January and December 2012.

Despite several advantages of TLG over SUVₘₐₓ, there is a debate regarding the optimal SUV threshold that should be used for TLG calculation. (See, e.g., References 18-20). Various relative or absolute thresholds have been suggested to calculate TLG. Most cut-offs can be derived from single publications, and none of them have been validated with phantom data. In fact, several studies have shown that the use of relative or absolute thresholds may not be accurate enough to delineate the metabolically active tumor volume for radiation therapy planning (See, e.g., References 31-23). Some of the difficulties with absolute or relative thresholds can be that they do not consider background activity.

Thus, it may be beneficial to provide an exemplary system, method and computer-accessible medium for quantifying FDG uptake on PET which can overcome at least some of the deficiencies described herein above.

### SUMMARY OF EXEMPLARY EMBODIMENTS

An exemplary system, method and computer-accessible medium for determining at least one characteristic of at least one tissue can be provided, which can include, for example receiving first information related to a volume of interest (VOI) of the tissue(s), receiving second information related to a histogram based on the tissue(s), determining a Gaussian fit of the histogram, and determining the characteristic(s) of the tissue(s) by at least partially subtracting or removing the Gaussian fit from the VOI. The Gaussian fit can be over a peak of the histogram. The histogram can be determined based on voxels of the VOI as a function of a standard uptake value of the tissue(s).

In some exemplary embodiments of the present disclosure, the histogram can be binned based on a Freedman-Diaconis rule. A width of the bin can be ΔSUV_{fd}=2 IQR(VOI) N^{-1/3}, where ΔSUV_{fd} can be a [18F]-Fluorodeoxyglucose standard uptake value, IQR(VOI) can be an interquartile range (VOI), and N can be a number of voxels in the IQR(VOI). The tissue(s) can be a tumor. The characteristic(s) can be a tumor activity, which can be a background subtracted lesion activity (BSL). The characteristic(s) can be a tracer uptake metric. A mean BSL can be determined based on a mode of the histogram. The Gaussian fit can be over a region defined by SUV_{BG}±SUV_{V}_{ₘₐₓ}_{/2}, where SUV_{BG} can be the mean BSL standard uptake value (SUV) and SUV_{V}_{ₘₐₓ}_{/2} can be an SUV of histogram bins located at a half maximum of the mode.

In some exemplary embodiments of the present disclosure, all negative values can be set to zero, and the Gaussian fit can be subtracted from the histogram based on the zero values. The second information can be generated using positron emission tomography. An image(s) of the tissue(s) can be generated based on the characteristic(s). A selection of the VOI can be received which can be selected by a user. Background data in the histogram can be approximated as a normal distribution. The VOI can be

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1A is an exemplary chart illustrating an exemplary sphere model with a lesion, causing a spillover, embedded in background activity according to an exemplary embodiment of the present disclosure;

FIG. 1B is an exemplary chart illustrating that the exemplary 42% threshold may not incorporate spillover into tumor activity according to an exemplary embodiment of the present disclosure;

FIG. 1C is an exemplary chart illustrating transposition of voxels into a histogram according to an exemplary embodiment of the present disclosure;

FIG. 2 is a set of exemplary charts illustrating 50 phantom chambers with three exemplary different PET quantification procedures according to an exemplary embodiment of the present disclosure;

FIG. 3 is a set of exemplary charts illustrating 50 phantom chambers with calculated TLG_{RC} and BSL versus the total injected activity according to an exemplary embodiment of the present disclosure;

FIG. 4A is an exemplary chart illustrating 25 lung lesions with a TLG_{2.5}≦50 ml*SUV, compared to the reference standard TLG_{RC} (e.g., CT volume*recovery coefficient corrected SUVₘₐₓ);

FIG. 4B is an exemplary chart illustrating 25 lung lesions with a TLG_{2.5}>50 ml*SUV, where BSL and TLG_{42%} can be compared to TLG_{2.5};

FIG. 5A is an exemplary MIP FDG PET image of a patient with a large lung tumor in the right upper lobe with an SUVₘₐₓ of 23.3 according to an exemplary embodiment of the present disclosure;

FIG. 5B is an exemplary image of an exemplary Axial slice of a tumor in the right upper lobe according to an exemplary embodiment of the present disclosure;

FIG. 5C is an exemplary histogram of the volume of interest of the images in FIGS. 5D-5F according to an exemplary embodiment of the present disclosure;

FIG. 5D is an exemplary image of all voxels with a SUV above 42% of SUVₘₐₓ representing TLG_{42%} according to an exemplary embodiment of the present disclosure;

FIG. 5E is an exemplary image of the volume of all voxels with an SUV above 2.5, representing TLG_{2.5} (e.g., 1810 ml*SUV) according to an exemplary embodiment of the present disclosure;

FIG. 5F is an exemplary image of all voxels above background (e.g., BSL 1969 ml*SUV) according to an exemplary embodiment of the present disclosure;

FIG. 6A is an exemplary MIP FDG PET image of a patient with a small lung tumor in the right upper lobe with a low FDG activity (e.g., SUVₘₐₓ of 1.5) and TLG_{2.5}<50 ml*SUV according to an exemplary embodiment of the present disclosure;

FIG. 6B is an exemplary image of an axial slice of a tumor in the right upper lobe according to an exemplary embodiment of the present disclosure;

FIG. 6C is an exemplary histogram of the volumes of interest illustrated in FIGS. 6D-6F according to an exemplary embodiment of the present disclosure;

FIG. 6D is an exemplary image of the volume covered by all voxels with a SUV above 42% of SUVₘₐₓ representing TLG_{42%} (e.g., 9.9 ml*SUV) according to an exemplary embodiment of the present disclosure;

FIG. 6E is an exemplary image illustrating that TLG_{2.5} can fail to measure any tumor activity (e.g., TLG_{2.5} 0 ml*SUV) according to an exemplary embodiment of the present disclosure;

FIG. 6F is an exemplary image of the activity of all voxels above background (e.g., BSL 15.2 ml*SUV), overestimating the reference activity for this lesion (e.g., TLG_{RC} 13.3 ml*SUV) only by about 14% according to an exemplary embodiment of the present disclosure;

FIG. 7 is an exemplary chart illustrating a plot of the data and the fit for GE Discovery STE PET/CT;

FIG. 8 is an exemplary flow diagram illustrating a method for determining a characteristic of a tissue according to an exemplary embodiment of the present disclosure; and

FIG. 9 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures.

### DETAILED DESCRIPTION OF EXEMPLARY EMBODIMENTS

An increase of use of molecular imaging probes as biomarkers in oncology can emphasize the need for robust and stable methods for quantifying tracer uptake on PET. An exemplary histogram-based procedure can be developed to calculate a new tracer uptake metric (e.g., BSL).

A thorax phantom and a PET-ACR quality assurance phantom can be scanned, e.g., 5 times with increasing FDG concentrations in the phantom chambers. VOIs can be placed over each phantom chamber. TLG can be calculated with a fixed threshold at SUV 2.5 TLG_{2.5} and a relative threshold at 42% of SUVₘₐₓ TLG_{42%}. The histogram for each VOI can be generated, and BSL can be calculated. BSL, TLG_{2.5} and TLG_{42%} can be compared against the total injected FDG activity using concordance correlation coefficients (“CCC”). Fifty consecutive patients with FDG-avid lung tumors were selected. TLG_{42%}, TLG_{2.5} and BSL were calculated and compared to the reference standard using CCC. The phantom results illustrate that an optimal reference standard for patients can be uptake dependent. For TLG_{2.5}≦50 ml*SUV (e.g., group 1) the reference uptake was found by multiplying the CT lung tumor volume by the recovery coefficient corrected SUVₘₐₓ. For lesions with a TLG_{2.5}>50 ml*SUV (e.g., group 2), TLG_{2.5} can be used as reference standard.

In both phantoms, the CCC between the injected activity and BSL were higher (e.g., 0.998) than for TLG_{42%} (e.g., 0.906) or TLG_{2.5} (e.g., 0.996). In the 50 lung lesions, BSL had a higher CCC as compared to the reference activity than did TLG_{42%} for both groups (e.g., CCC group 1: 0.680 vs. 0.297 and group 2: 0.987 vs. 0.799) and was higher than the TLG_{2.5} in group 1 (e.g., CCC 0.680 vs. 0.589).

The exemplary histogram based BSL correlated better with the injected activity in both phantom studies. Additionally, in lung tumors, the BSL activity can be superior in assessing the lesion activity as compared to commonly applied TLG quantification procedures. BSL therefore is an important new tool to increase the accuracy of molecular imaging quantification procedures.

### Exemplary of Phantom Studies

The BSL, TLG42% and TLG2.5 can be compared in two phantom studies with a wide range of different chamber sizes and activity concentrations. The true activities can be calculated for each chamber and acquisition by multiplication of the known chamber volume with the injected FDG concentration, and can be referred to as the total injected activity (“TIA”). TIA can be the reference standard to compare the histogram based BSL with TLG2.5 and TLG42%.

The exemplary CT data can be used to calculate a PET independent tumor volume that can be used as an alternative reference when using the recovery coefficient and the SUVmax to estimate the total tumor uptake (“TLGRC”). TLGRC can be validated against TIA in the exemplary phantoms. TLGRC can be the product of the partial volume corrected maximum activity concentration and the known volume of the phantom chambers.=

### Exemplary Estimating the Recovery Coefficients:

According to an exemplary embodiment of the present disclosure, recovery coefficients can be estimated by a least squares fit of phantom data using a two parameter fitting function given by, for example:

RC=x₁ log V+x₂

where RC can be the recovery coefficient, V can be the volume (ml), and x_{1,2} can be the fitting coefficients. Using data acquired on an GE DSTE PET/CT system with an IEC phantom with some additional inserts these coefficients can determined to be, for example:

RC=0.129 log V+0.535

RC=x₁ log V+x₂

Based on the results of the phantom studies, surrogate references can be defined for the total activity estimation in lung tumors in patients. For lesions with a high FDG activity, TLG2.5 can be expected to yield accurate results as compared to TIA. However, TLGRC can be restricted to homogeneous lesions, and therefore, in real tumors, can be more suitable for smaller volumes where PET images of tumors can be more homogenous. Therefore, both quantification metrics can be validated against TIA in the two phantoms to determine the appropriate cutoff point that can minimize the relative error for both TLGRC and TLG2.5 as compared to TIA. For all lesions with a TLG below this threshold, TLGRC can serve as reference. For the lesions above the threshold TLG2.5 can be the reference standard. TLG42%, TLGRC, TLG_{2.5} and BSL can then be compared to the surrogate reference.

### Exemplary BSL Calculation

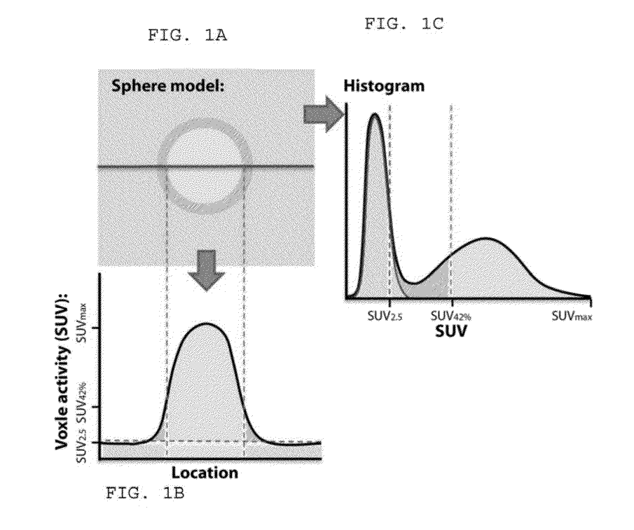

BSL is an exemplary histogram based procedure that can determine the tumor activity by subtraction of a Gaussian fit over the peak of the histogram from the total VOI. The exemplary histograms can represent the voxels of a VOI as a function of SUV, and can be binned via the Freedman-Diaconis rule. (See, e.g., Reference 24; Appendix 2). The mean background activity of the surrounding tissue, SUV_{BG}, can be estimated by the mode of the histogram. A fitting region can then be defined as SUV_{BG}±SUV_{V}_{ₘₐₓ}_{/2}, where SUV_{V}_{ₘₐₓ}_{/2} can be the SUV of the histogram bins located at the half maximum of the mode. The Gaussian fit over this region (see, e.g., FIG. 1C) can represent the background activity (see, e.g., FIGS. 1A and 1B) and can be subtracted from the histogram after setting all negative values to zero. BSL can be the sum of the remaining voxels in the subtracted histogram from SUV_{BG+2σ} to SUVₘₐₓ. (See, e.g., FIG. 1C).

BSL can be a variant of the TLG measurement. BSL can be estimated via subtracting a Gaussian fitted to the background region in a histogram built as a function of volume versus SUV within a selected VOI. BSL can utilize a user specified VOI, a properly binned histogram, and a restricted fitting region within the histogram. Each of these is discussed in detail below.

### Exemplary Histogram Bin Size:

In order to perform a histogram analysis on a lesion the VOI can be defined such that it encloses both the lesion and the highest uniform uptake component of the background as the dominant portion of background. The SUV value for the ith voxel in the VOI can thus be given by, for example:

$$VOI ( {SUV}_{i} ) = \{ \begin{matrix}{SUV}_{i} \\ 0\end{matrix} , {SUV}_{i} ∈ VOI .$$

Given the variability in size and heterogeneity, the SUV values can be binned in an appropriate manner that can be robust to non-normally distributed data. In this exemplary case, the Freedman-Diaconis rule can be used (see. e.g., Reference 24), which can give the bin width as, for example:

ΔSUV_{fd}=2IQR(VOI)N^{-1/3}.

Here, ΔSUV_{fd} can be the [¹⁸F]-Fluorodeoxyglucose SUV for, N can be the number of voxels in the VOI, interquartile range (“IQR”) (VOI) can be the interquartile range of the data in the VOI. This gives the total number of bins as, for example:

$${N}_{bins} = ⌈ \frac{{SUV}_{max} - {SUV}_{min}}{Δ {SUV}_{fd}} ⌉$$

where ┌•┐ can be the greatest integer function, and, for example:

$$Δ SUV = \frac{{SUV}_{max} - {SUV}_{min}}{{N}_{bins}} .$$

The kth bin (kε[0, N_{bins}−1]) of the histogram can be given by, for example:

SUVₖ=kΔSUV+SUVₘᵢₙ

and the volume of the k^{th} bin can be given by the histogram function, for example:

Vₖ=H(SUVₖ,ΔSUV).

### Exemplary Defining the Fitting Region:

Histograms of tumor VOI can have a characteristic asymmetric shape, with a peak representing the homogeneous background activity and a tail representing the far more heterogeneous lesion activity. The exemplary system, method and computer-accessible medium, according to an exemplary embodiment of the present disclosure, can fit the background portion of the histogram with a Gaussian fit. To do this, it can be assumed that the background data in the VOI's histogram can be reasonably approximated as being normally distributed. This holds if the background region can be sampled near the mode of its distribution. For a reliable fit, the region can be restricted to the area around the mode of the histogram. It has been previously shown that that the mode of a histogram from a VOI surrounding a lesion can be a reasonable estimate of the background region's mean. Thus, the index of the histogram at the maximum of the number of bins can be given by, for example:

$${k}_{bkg} = ] \frac{mode ( H ( SUV , Δ SUV ) ) - {SUV}_{min}}{Δ SUV} [$$

where ]•[ can be the rounding function and the SUV at that index can be given by, for example:

SUV_{bkg}=SUVₖ_{_{bkg}}

which can be an estimate of the mean of the background. As a result, the mean background and its interquartile range give the indices of the fitting region as, for example:

$${k}_{fit} ∈ [ {k}_{bkg} - ⌈ \frac{IQR ( VOI )}{Δ SUV} ⌉ , {k}_{bkg} + ⌈ \frac{IQR ( VOI )}{Δ SUV} ⌉ ] .$$

### Exemplary Background Fitting

Fitting a Gaussian function to the histogram over the fitting region can be done, by minimizing, the objective function defined as

$$Φ ( a , μ , σ ) = ‖ \frac{{V}_{{k}_{fit}} - aN ( {SUV}_{fit} \vert μ , σ )}{{\sum }_{{k}_{fit}}^{ } {V}_{k}} ‖$$

where ∥•∥ represents the norm of the interior (e.g., a 2-norm) and the parameters a, μ, and σ (e.g., scale, mean, and spread of the Gaussian, N(•)) can be all constrained to be positive. Hence, [a*μ*σ*]=arg min_{[a μ σ]} Φ scan be the fitting parameters that minimize the objective function. Thus, the indices of the fitted mean and spread of the background can be given by, for example:

$${k}_{{μ}^{*}} = ] \frac{{μ}^{*} - {SUV}_{min}}{Δ SUV} [$$

and, for example:

$$Δ {k}_{{σ}^{*}} = ⌈ \frac{{σ}^{*}}{Δ SUV} ⌉ .$$

### Exemplary Background—Subtracted Lesion Estimation:

The background-subtracted lesion for glycolic uptake can be the sum of the activity in the histogram after subtraction of the fitted volume representing the background activity. This can be defined as the summed positive valued portion of the histogram for k≧k_{μ*}+2Δk_{σ*}. This can be given by, for example:

$${BSL}_{g} = {\sum }_{k = {k}_{{μ}^{*}} + 2 Δ {k}_{{σ}^{*}}}^{{N}_{bin}} {\{ {SUV}_{k} ( {V}_{k} - {a}^{*} N ( {SUV}_{k} \vert {μ}^{*} , {σ}^{*} ) ) \}}^{+}$$

where {•}⁺ can represent the function that passes positive values, all negative values can be zero.

### Exemplary Phantom Details

Two exemplary phantoms can be used: the Society of Nuclear Medicine Clinical Trials Network (“SNM-CTN”) anthropomorphic thorax phantom, and the American College of Radiology (“ACR”) (e.g., flangeless Esser PET Phantom™) cylindrical phantom with separately fillable cylinders. The SNM-CTN phantom can be initially filled according to the SNM-CTN instructions and scanned with 555 MBq (e.g., 15 mCi) entered as the injected dose, 163 cm (e.g., 64 inches), and 63 kg (e.g., 140 lbs.) for the patient height and weight (e.g., the actual activity concentrations and ratios are given in Table 1). In accordance with ACR guidelines, the patient weight and injection can be entered as a 70 kg patient with a 444 MBq (e.g., 12 mCi) injection (e.g., the actual activity concentrations and ratios are given in Table 1). The residual activities can be accounted for both phantoms. In each of the four subsequent scans, the fillable chambers can be drained and refilled with increasing activity concentrations. (See, e.g., Table 1 for the hot sphere activities and imaging times).

### Exemplary Patient Selection, Preparation and Acquisition:

Fifty consecutive patients fulfilling the following inclusion criteria between January and March 2011 can be retrospectively identified: (i) upper lobe lung tumors, (ii) FDG PET/CT scan can be performed using a GE DSTE PET/CT system (e.g., GE Medical Systems, Wisconsin). Scans can be acquired approximately 1-hour post injection with a nominal 444 MBq (e.g., 12 mCi) of FDG. A low-dose, attenuation correction, CT scan (e.g., 120-140 kV, approximately 80 mA) can be acquired. This can be followed by acquisition of PET emission images form the pelvis to the skull for 3 minutes per bed position with an 11-slice overlap.

### Exemplary Image Reconstruction

The exemplary image reconstruction settings can be identical for both the phantom and patient acquisitions. The images can be reconstructed using exemplary clinical settings: OSEM with 2 iterations with 20 subsets and 6.3 mm post reconstruction trans-axial filtering and three-point [1 2 1] smoothing (e.g., Heavy) along the z-axis. Corrections to the images can be applied (e.g., attenuation, normalization, scatter, randoms from singles, decay and dead time).

### Exemplary Phantom Data Analysis

In the phantoms, a VOI can be drawn around each chamber. The CT attenuation scans of the SNM-CTN phantom can reveal air bubbles of varying sizes in chamber number 4. This chamber can be excluded from any further analysis. A total of 10 chambers can be analyzed in five scans with increasing activities in the chambers. BSL, TLG42% and TLG2.5 can be compared to TIA.

### Exemplary Patient Data Analysis

For 50 patients, one lesion can be selected, and a VOI can be drawn around the tumor. VOI size can be slightly bigger than the tumor being scanned. For lesions with heterogeneous background (e.g., tumors abutting lung and mediastinal tissue) VOIs can be adjusted to ensure that more of the background tissue with higher FDG activity can be included (e.g., mediastinum). Selection criteria can include lesions that are well delineated on the low dose CT for attenuation correction that there is FDG activity higher than background and that other significant abnormalities on CT (e.g., pulmonary atelectasis or consolidation) can be absent near the tumor. CT Volume of each lesion can be determined using a manual volume segmentation tool (e.g., from commercially available software e.g., TeraRecon, Inc. Foster City, Calif. (USA)).

### Exemplary Statistical Analysis:

The correlation of TLG42%, TLG2.5 and BSL with TIA in the phantom or the surrogate reference for the lung tumor data can be calculated with several procedures. A least-squares line fit with zero-intercept slope (e.g., a) can be calculated for each TLG or BSL measure versus the reference values. For linearity the slope (e.g., s) and the correlation (e.g., R2) can be assessed. Further, the concordance correlation coefficient (e.g., CCC) (see, e.g., Reference 25) can be calculated and its significance for each comparison to reference.

### Exemplary Results

### Exemplary BSL Validation in Phantoms

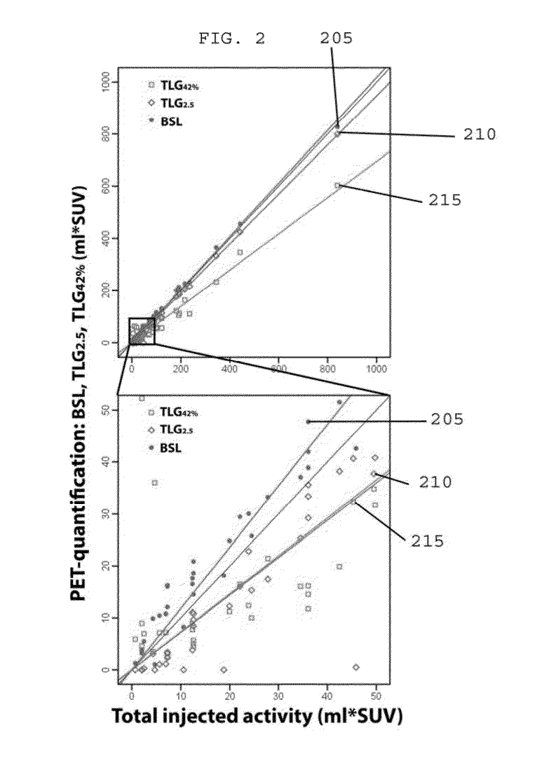

For all lesions, the correlation between TIA and BSL, TLG42% and TLG2.5 can be similar, with CCCs 0.998, 0.906 and 0.996 respectively. A cut-off to minimize the relative error between TLG2.5 and TIA can be between 40-60 ml*SUV. Therefore 50 ml*SUV can be selected as the cut-off. This value can have the added benefit of splitting the exemplary patient population in half. For lesions with a TIA≦50 ml*SUV, the correlation can still be very good for BSL (e.g., CCC=0.933), but only moderate for TLG2.5 (e.g., CCC=0.761). (See, e.g., Table 4). TLG42% had a lower CCC for both groups TIA≦50 and >50 ml*SUV with 0.350 and 0.873, respectively. The slopes can reveal a slight overestimation of BSL (element 205 of FIG. 2) versus TIA with a slope of 1.015-1.189, with high R2 values 0.876-0.999, whereas TLG2.5 (element 210 of FIG. 2) underestimated chambers with a TIA≦50 ml*SUV (e.g., 0.727) with more variance (e.g., R2=0.876), but can be accurate for chambers with TIA>50 ml*SUV (e.g., s=0.952, R2=0.999). TLG42% (element 215 of FIG. 2) can underestimate the activity in both groups with slopes between 0.694-0.731 (e.g., R2 values 0.0.511-0.986). (See e.g., FIG. 2) The results for the ACR phantom chambers are given in Table 2, including the relative difference between the TIA and the three PET-quantification metrics (e.g., TLG42%, TLG2.5 and BSL). Table 3 illustrates the values for the chambers in the SNM thorax phantom.

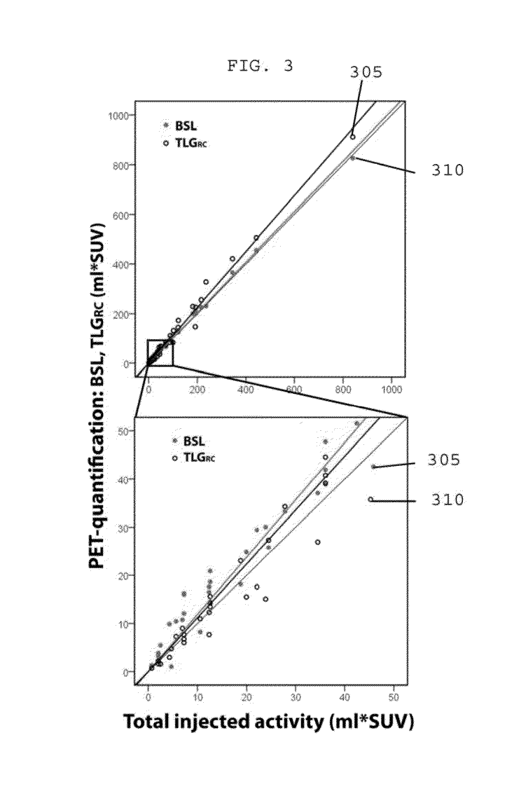

FIG. 3 shows a set of exemplary charts illustrating 50 phantom chambers with calculated TLGRC (element 305) and BSL (element 310) versus the total injected activity. TLGRC is shown as a volume and SUVmax based measurement that can be validated as an alternative reference for lesions with FDG uptake under 50 ml*SUV. The exemplary Phantom results confirmed a high correlation of TLGRC with TIA

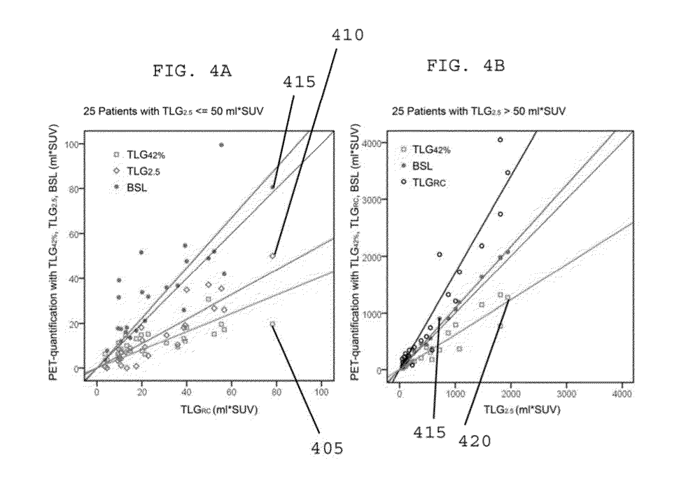

FIG. 4A illustrates an exemplary chart illustrating showing 50 patients with 25 lung lesions with a TLG2.5≦50 ml*SUV, compared to the reference standard TLGRC (e.g., CT volume*recovery coefficient corrected SUVmax). As shown in FIG. 4A, TLG42% (element 405) and TLG2.5 (element 410) underestimated the tumor activity as compared to BSL (element 415).

FIG. 4B illustrates an exemplary chart illustrating 50 patients with 25 lung lesions with a TLG2.5>50 ml*SUV, where BSL (element 415) and TLG42% (element 420) compared to TLG2.5. As shown in FIG. 4B, BSL had an almost perfect correlation (CCC=0.987), while TLG42% underestimated the tumor activity

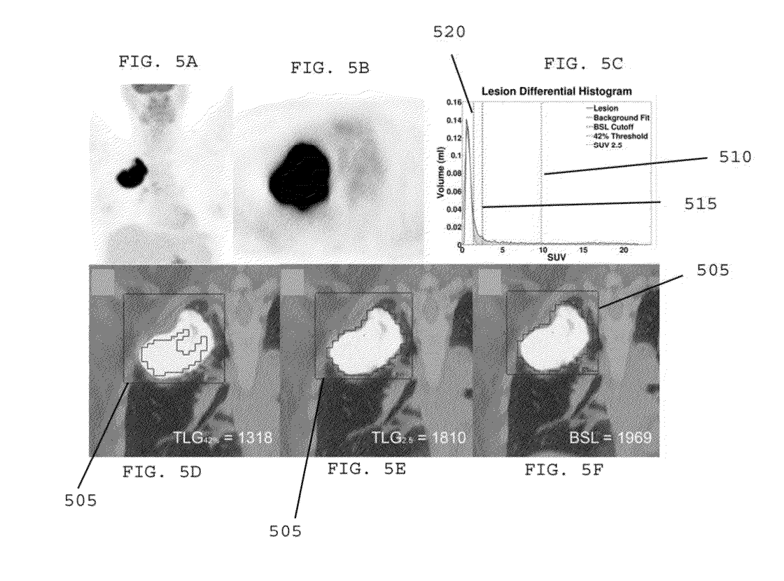

FIG. 5A shows an exemplary Maximum Intensity Projection (“MIP”) FDG PET image of a patient with a large lung tumor in the right upper lobe with an SUVmax of 23.3. FIG. 5B is an exemplary image of an exemplary axial slice of a tumor in the right upper lobe. FIG. 5C is an exemplary histogram of the volume of interest 505 of the images in FIGS. 5D-5F. FIG. 5C illustrates the threshold lines for TLG42% (element 510), TLG2.5 (element 515) and the cut off for BSL (element 520). BSL can be represented by the sum of all yellow voxels

FIG. 5D illustrates an exemplary image of all voxels with a SUV above 42% of SUVmax representing TLG42%. FIG. 5E is an exemplary image of the volume of all voxels with an SUV above 2.5, representing TLG2.5 (e.g., 1810 ml*SUV). FIG. 5F illustrates an exemplary image of all voxels above background (e.g., BSL 1969 ml*SUV).

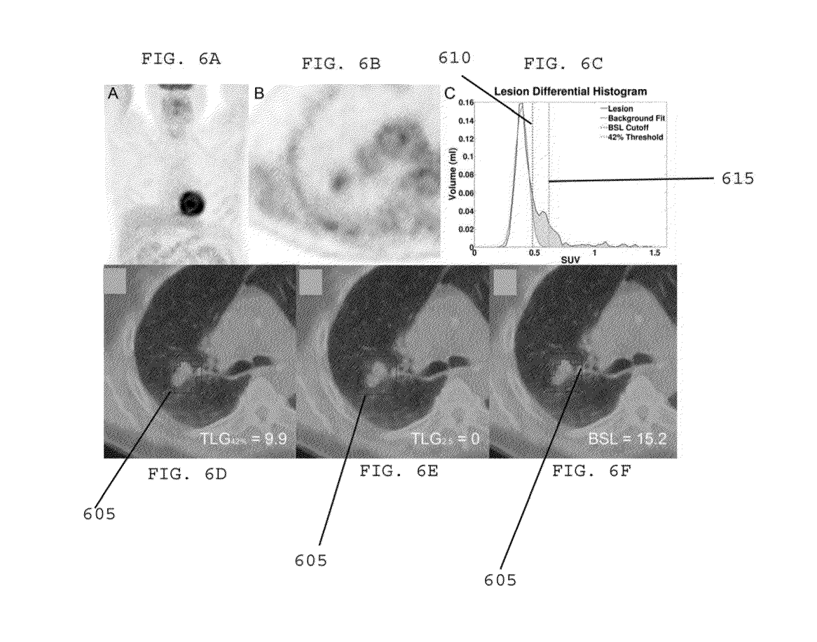

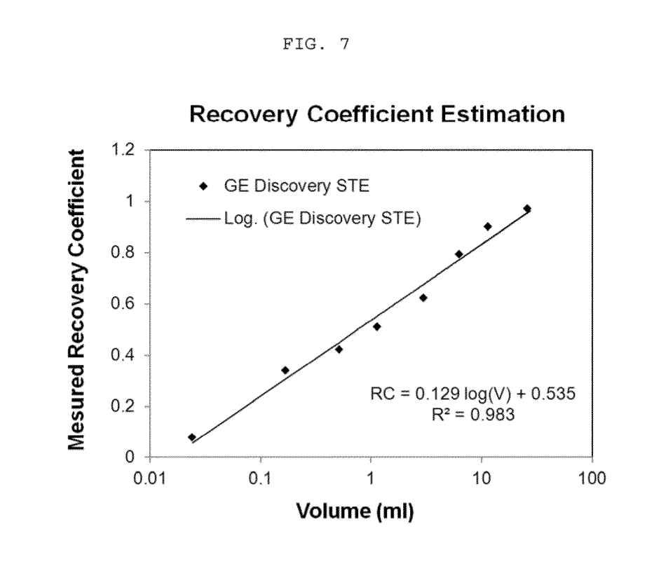

FIG. 6A shows an exemplary MIP FDG PET image of a patient with a small lung tumor in the right upper lobe with a low FDG activity (e.g., SUVmax of 1.5) and TLG2.5<50 ml*SUV. FIG. 6B illustrates an exemplary image of an axial slice of a tumor in the right upper lobe. FIG. 6C shows an exemplary histogram of the volumes of interest 605 illustrated in FIGS. 6D-6F. FIG. 6C illustrates both threshold lines for TLG42% (element 610) and the cut off for BSL (element 615). BSL can be represented by the sum of all yellow voxels in the histogram

FIG. 6D shows an exemplary image of the volume covered by all voxels with a SUV above 42% of SUVmax representing TLG42% (e.g., 9.9 ml*SUV). FIG. 6E illustrates an exemplary image illustrating that TLG_{2.5} can fail to measure any tumor activity (e.g., TLG_{2.5} 0 ml*SUV). FIG. 6F shows an exemplary image of the activity of all voxels above background (e.g., BSL 15.2 ml*SUV), overestimating the reference activity for this lesion (e.g., TLG_{RC} 13.3 ml*SUV) only by about 14%.

### Exemplary Recovery Coefficient Validation in Phantoms

The volume and recovery coefficient corrected SUVₘₐₓ based FDG quantification correlated very well with TIA for both lesions with a TIA below or over 50 ml*SUV (e.g., CCC=0.931-0.984). There can be a slight overestimation of the cylindrical lesions, since the exemplary procedure can be developed for spherical lesions (See, e.g., FIG. 3; Table 5).

### Exemplary Validation of BSL in Lung Tumors Against TLGRC and TLG2.5

Of the 50 selected patients, 25 had a TLG2.5≦50 ml*SUV (see, e.g., Table 5) and 25 can be above this threshold (see, e.g., Table 6). Thus, the patients can be separated into two groups. For group 1 (e.g., TLG2.5≦50 ml*SUV) the PET quantification metrics can be compared with TLGRC. (See, e.g., FIG. 4A). Both TLG2.5 and TLG42% underestimated the reference activity (e.g., s=0.548 and 0.408, respectively), whereas BSL can be very close to one (e.g., s=1.117), and only slightly higher than TLGRC. (See, e.g., Table 7). BSL can also have the highest correlation (e.g., CCC 0.68) as compared to TLGRC.

For group 2 (e.g., TLG2.5>50 ml*SUV), TLG2.5 can serve as the reference activity, TLGRC overestimated the activities of the lesions with high activity substantially (e.g., s=1.705), whereas TLG42% underestimated the reference activity (e.g., s=0.618). BSL and TLG2.5 had an excellent correlation (e.g., CCC 0.987) with a slope of s=1.084. (See, e.g., FIG. 4B; Table 7).

### Exemplary Discussion

The exemplary results of the phantom analysis can illustrate that the BSL can be superior in assessing FDG uptake on PET compared to the two commonly applied TLG quantification procedures. The common procedures for TLG assessment may not consider background activity and can be based on measures that have systematic errors; TLG42% underestimates the activity in lesions with a high SUVₘₐₓ (see, e.g., FIG. 5), whereas TLG2.5 underestimates the activity in lesions with relatively low FDG activity. (See, e.g., FIG. 6). It can also be shown that the BSL can be correlated slightly better with the true injected activity than the recovery coefficient based TLGRC.

The optimal cut-off for TLG assessment has been extensively investigated in the literature. Several studies have evaluated various relative (e.g., 25%, 50% or 75% of SUVₘₐₓ) 20 or absolute thresholds (e.g., SUV 2.5, 3, 3.5 or 4). (See, e.g., References 18 and 19). Some of these thresholds have been shown to be superior to 42% in certain tumor entities, however all thresholds can be based on SUVₘₐₓ, which in itself can be shown to have an intrinsic variability of 20-30%. (See, e.g., References 6-8). In addition, physiological tracer uptake can vary in different anatomical locations, and this can affect the use of absolute thresholds for delineating malignant from benign diseases.

Prior studies have suggested histogram analysis can be useful for separating different parts of a tumor into variable categories. (See e.g., Reference 26). However, to use a histogram based analysis to calculate the background subtracted lesion activity, as an equivalent to TLG, has not been examined.

A subtraction of a Gaussian fit to the peak of a histogram to determine BSL can be incorporated into the exemplary system, method and computer-accessible medium. To determine the robustness of the procedure, two phantom studies were performed with increasing tumor to background ratios and chamber sizes. An important question for any PET segmentation procedure can be the accuracy in patients, where the activity distribution can be more heterogeneous and the lesion to background boundaries less well defined.

For lesions with low FDG uptake, and a volume definable by CT, TLGRC can be used as a reference standard, since the exemplary phantom data can show a good correlation with TIA. Small lesions can be more likely to have a homogeneous FDG uptake on imaging due to the scanners resolution masking the true heterogeneity. Therefore, it can be assumed that TLGRC can be considered as a reasonable approximation of the total FDG uptake in small lesions, provided the volume can be well defined on CT. For large lesions however, the heterogeneity of the tumor, with large areas of lower activity than the measured SUVₘₐₓ can lead to an overestimation of the total tumor burden with TLGRC. Therefore two different surrogates can be used as references for total tumor burden in patients.

The need for considering the background tracer uptake when quantifying trace uptake in tumor lesions has also been previously mentioned and different solutions can be suggested; either by incorporating a standardized background activity for each anatomical region (e.g., bone, soft tissue) (see, e.g., Reference 27) or by placing separate VOI over undiseased tissue adjacent to tumors. (See, e.g., References 22 and 28). The latter can be an accurate approach. However placing an additional “background” VOI for every tumor VOI can substantially increase workload, particularly in patients with extensive disease. With the histogram based BSL segmentation, a procedure can be developed to subtract background activity from the tumor VOI without any further measurements or assumptions. However, the resulting BSL may not correspond to an anatomical volume, but instead can represent the total lesion activity including also activity measured outside the actual tumor border from tumor spill-out. Further, the subtraction of the Gaussian fit in the histogram can mean that there may not be a sharp threshold to distinguish which specific voxels can be counted and which may not be counted.

Tumor volume determination with PET can be intrinsically difficult due to the heterogeneity of tumor uptake, the variety of different tumor shapes, the variability of the surrounding background activity, and the limited resolution of the images. However, for metabolic tumor quantification, the total lesion uptake can be more important than the anatomical volume. The accuracy of the total uptake measurement over delineation of the true tumor volume can be focused on. Therefore, no spillover correction can be applied, with the assumption that this activity originated from the lesion itself. Indeed, the simple BSL correlated significantly better with the known injected amount of FDG activity in both phantom studies when compared to the two most commonly used procedures to determine TLG and even when compared to TLGRC.

It can be difficult to validate any procedure for tumor quantification against the published TLG due to the lack of a true gold standard. As an alternative, the use of two different surrogate references can be methodically suboptimal, however when looking at the exemplary phantom results, it can be concluded that TLGRC can be a suitable reference for homogeneous lesions with a known volume, and the TLG2.5 can serve as an accurate reference for lesions with a TLG above 50 ml*SUV.

Furthermore, the idea of a recovery coefficient correction to estimate the true activity in lung nodules has been previously known. (See. e.g., Reference 29). This value however, can depend on scanner specific properties such as spatial resolution. (See. e.g., Reference 30). For the exemplary analysis, an RC value can be used that can be determined on exemplary scanners. In appendix 1, the data used to establish this value and the RC procedure itself can be explained. Doing the same calculation with the published RC values for the older scanner generations or different venders can impair the results; therefore, a single value can be used.

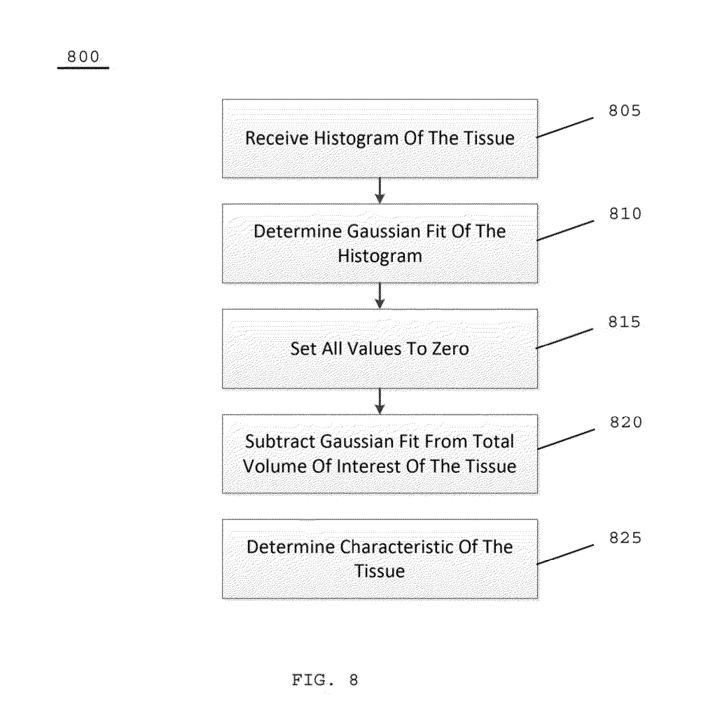

FIG. 8 shows an exemplary diagram illustrating an exemplary method 800 for determining a characteristic of a tissue according to an exemplary embodiment of the present disclosure. For example, at procedure 805, information related to a histogram of the tissue can be received. At procedure 810, the Gaussian fit of the histogram can be determined, and the values of the volume of interest can be set to zero at procedure 815. At procedure 820, the Gaussian fit can be subtracted from the total volume of interest of the tissue, and the characteristic of the tissue can be determined at procedure 825.

### Exemplary Conclusion

The exemplary BSL procedure to quantify tumor uptake with simple histogram analysis can be a reliable and robust tool for FDG uptake quantification in both phantom studies and 50 patients. Looking at the increasing use of TLG in the literature, this can become an important new procedure to increase the accuracy of molecular imaging tumor quantification.

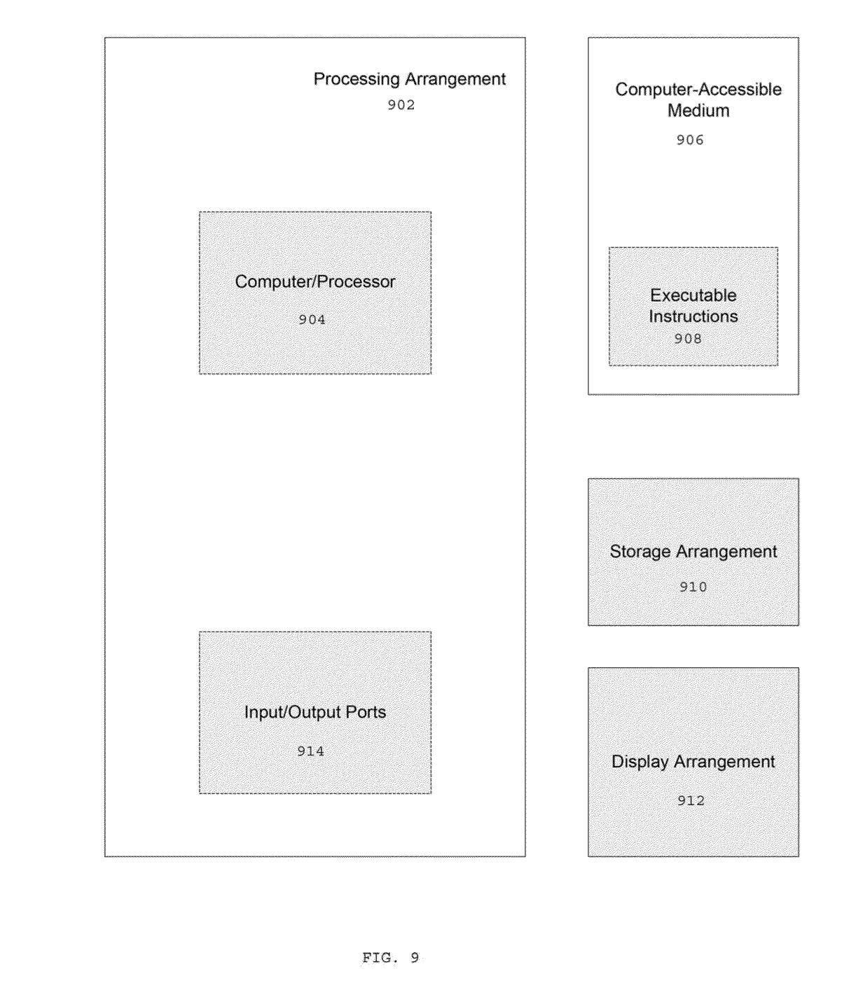

FIG. 9 shows a block diagram of an exemplary embodiment of a system according to the present disclosure. For example, exemplary procedures in accordance with the present disclosure described herein can be performed by a processing arrangement and/or a computing arrangement 902. Such processing/computing arrangement 902 can be, for example, entirely or a part of, or include, but not limited to, a computer/processor 904 that can include, for example, one or more microprocessors, and use instructions stored on a computer-accessible medium (e.g., RAM, ROM, hard drive, or other storage device).

As shown in FIG. 9, for example, a computer-accessible medium 906 (e.g., as described herein above, a storage device such as a hard disk, floppy disk, memory stick, CD-ROM, RAM, ROM, etc., or a collection thereof) can be provided (e.g., in communication with the processing arrangement 902). The computer-accessible medium 906 can contain executable instructions 908 thereon. In addition or alternatively, a storage arrangement 910 can be provided separately from the computer-accessible medium 906, which can provide the instructions to the processing arrangement 902 to configure the processing arrangement to execute certain exemplary procedures, processes and methods, as described herein above, for example.

Further, the exemplary processing arrangement 902 can be provided with or include an input/output arrangement 914, which can include, for example, a wired network, a wireless network, the internet, an intranet, a data collection probe, a sensor, etc. As shown in FIG. 9, the exemplary processing arrangement 902 can be in communication with an exemplary display arrangement 912, which, according to certain exemplary embodiments of the present disclosure, can be a touch-screen configured for inputting information to the processing arrangement in addition to outputting information from the processing arrangement, for example. Further, the exemplary display 912 and/or a storage arrangement 910 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### Exemplary Tables

TABLE 1
Acquisition times and activity concentrations
for the SNM and ACR phantom tests.

| | | SNM Phantom | | |
|---|---|---|---|---|
| Scan | Scan | Hot Cylinders | Background | Activity |
| Number | Time | (kBq/cc) | (kBq/cc) | Ratios |
| 1 | 20:38 | 23.7 | 7.7 | 3.1 |
| 2 | 20:46 | 62.1 | 6.5 | 9.5 |
| 3 | 20:39 | 98.5 | 5.3 | 18.7 |
| 4 | 20:49 | 244.6 | 4.6 | 53.1 |
| 5 | 20:53 | 424.7 | 4.1 | 104.0 |
| Chamber volumes: 0.18, 3 × 0.52, 1.4, and 4.2 ml | | | | |
| | | ACR Phantom | | |
| Scan | Scan | Hot Cylinders | Background | Activity |
| Number | Time | (kBq/cc) | (kBq/cc) | Ratios |
| 1 | 19:36 | 11.4 | 5.9 | 1.9 |
| 2 | 19:57 | 32.3 | 5.2 | 6.2 |
| 3 | 20:15 | 51.0 | 4.7 | 10.9 |
| 4 | 20:32 | 94.2 | 4.2 | 22.5 |
| 5 | 20:51 | 158.6 | 3.7 | 42.8 |

Chamber volumes: 2, 4.5, 8.5, and 28.5 ml

TABLE 2
ACR Phantom with 4 hot lesions, scanned 5 times with increasing lesion to
background ratios. The background can have an activity of SUV 1, filled
according to the ACR guidelines. TIA calculated with the CT-volume and the
injected concentration can be the reference for validation of TLG and BSL.

| | Scan | CT Volume | | | Ref Diff | | Ref Diff | | Ref Diff |
|---|---|---|---|---|---|---|---|---|---|
| Nr. | Nr. | (cm [symbol] ) | TIA | TLG_{42%} | TLG_{42%} | TLG_{2.5} | TLG_{2.5} | BSL | BSL |
| 1 | 1 | 2.0 | 4.7 | 36.0 | 667% | 0.0 | −100% | 1.0 | −78% |
| | 2 | 2.0 | 12.4 | 8.9 | −28% | 3.8 | −69% | 16.5 | 33% |
| | 3 | 2.0 | 22.1 | 16.5 | −26% | 16.0 | −28% | 29.5 | 33% |
| | 4 | 2.0 | 45.3 | 32.3 | −29% | 40.7 | −10% | 56.1 | 24% |
| | 5 | 2.0 | 86.0 | 39.8 | −54% | 76.3 | −11% | 93.9 | 9% |
| 2 | 1 | 4.5 | 10.6 | 62.6 | 492% | 0.0 | −100% | 8.2 | −22% |
| | 2 | 4.5 | 27.8 | 21.4 | −23% | 17.4 | −37% | 33.3 | 19% |
| | 3 | 4.5 | 49.7 | 31.7 | −36% | 40.8 | −18% | 59.2 | 19% |
| | 4 | 4.5 | 101.9 | 57.7 | −43% | 96.5 | −5% | 114.3 | 12% |
| | 5 | 4.5 | 193.5 | 111.6 | −42% | 185.5 | −4% | 201.5 | 4% |
| 3 | 1 | 8.0 | 18.8 | 58.2 | 210% | 0.0 | −100% | 18.2 | −3% |
| | 2 | 8.0 | 49.5 | 34.8 | −30% | 37.8 | −24% | 64.2 | 30% |
| | 3 | 8.0 | 88.4 | 56.6 | −36% | 80.1 | −9% | 101.3 | 15% |
| | 4 | 8.0 | 181.2 | 121.7 | −33% | 176.2 | −3% | 200.6 | 11% |
| | 5 | 8.0 | 344.1 | 232.2 | −33% | 332.6 | −3% | 364.9 | 6% |
| 4 | 1 | 19.6 | 45.8 | 61.6 | 34% | 0.5 | −99% | 42.6 | −7% |
| | 2 | 19.6 | 120.9 | 92.5 | −23% | 97.7 | −19% | 130.6 | 8% |
| | 3 | 19.6 | 215.9 | 163.1 | −24% | 204.6 | −5% | 226.1 | 5% |
| | 4 | 19.6 | 442.3 | 346.0 | −22% | 424.4 | −4% | 455.3 | 3% |
| | 5 | 19.6 | 840.0 | 602.9 | −28% | 799.4 | −5% | 827.0 | −2% |

[symbol]

indicates data missing or illegible when filed

TABLE 3
SNM Phantom with 7 hot lesions, scanned 5 times with increasing lesion to
background ratios (e.g., Lesion 4 excluded due to irregular filling). TIA calculated
with the CT-volume and the injected concentration can be the reference for
validation of TLG and BSL.

| | | CT | | | | | | | |
|---|---|---|---|---|---|---|---|---|---|
| | | Volume | | | Ref Diff | | Ref Diff | | Ref Diff |
| Nr. | Scan Nr. | (cm³) | TIA | TLG_{42%} | TLG_{42%} | TLG_{2.5} | TLG_{2.5} | BSL | BSL |
| 1 | 1 | 1.4 | 5.6 | 7.1 | 27% | 1.1 | −80% | 10.5 | 88% |
| | 2 | 1.4 | 19.9 | 11.2 | −44% | 12.2 | −39% | 24.8 | 25% |
| | 3 | 1.4 | 34.5 | 16.1 | −53% | 25.3 | −27% | 37.1 | 8% |
| | 4 | 1.4 | 99 | 53.9 | −46% | 100.4 | 1% | 116.1 | 17% |
| | 5 | 1.4 | 191.4 | 104.1 | −46% | 187.3 | −2% | 212.7 | 11% |
| 2 | 1 | 0.5 | 2.1 | 4.6 | 119% | 0 | −100% | 3.6 | 71% |
| | 2 | 0.5 | 7.3 | 3.1 | −58% | 3.4 | −53% | 16.3 | 123% |
| | 3 | 0.5 | 12.6 | 5.1 | −60% | 9.7 | −23% | 18.6 | 48% |
| | 4 | 0.5 | 36.1 | 14.5 | −60% | 33.4 | −7% | 42 | 16% |
| | 5 | 0.5 | 69.8 | 33 | −53% | 65.1 | −7% | 74.4 | 7% |
| 3 | 1 | 0.2 | 0.7 | 5.9 | 743% | 0 | −100% | 1.3 | 86% |
| | 2 | 0.2 | 2.5 | 7 | 180% | 0.3 | −88% | 5.5 | 120% |
| | 3 | 0.2 | 4.3 | 3.5 | −19% | 3.1 | −28% | 9.9 | 130% |
| | 4 | 0.2 | 12.4 | 7.8 | −37% | 11 | −11% | 17.6 | 42% |
| | 5 | 0.2 | 23.9 | 12.4 | −48% | 22.8 | −5% | 30.1 | 26% |
| 5 | 1 | 0.5 | 2.1 | 52.2 | 2386% | 0 | −100% | 3.9 | 86% |
| | 2 | 0.5 | 7.3 | 3.2 | −56% | 3.2 | −56% | 16 | 119% |
| | 3 | 0.5 | 12.6 | 5.7 | −55% | 10.8 | −14% | 20.9 | 66% |
| | 4 | 0.5 | 36.1 | 16.1 | −55% | 35.6 | −1% | 47.8 | 32% |
| | 5 | 0.5 | 69.8 | 30.6 | −56% | 68.5 | −2% | 86.6 | 24% |
| 6 | 1 | 0.5 | 2.1 | 8.9 | 324% | 0 | −100% | 3.2 | 52% |
| | 2 | 0.5 | 7.3 | 2.6 | −64% | 2.3 | −68% | 12.1 | 66% |
| | 3 | 0.5 | 12.6 | 4.5 | −64% | 8.4 | −33% | 14.5 | 15% |
| | 4 | 0.5 | 36.1 | 11.7 | −68% | 29.3 | −19% | 38.9 | 8% |
| | 5 | 0.5 | 69.8 | 30.4 | −56% | 59.4 | −15% | 65.4 | −6% |
| 7 | 1 | 1.8 | 6.9 | 7.2 | 4% | 1.1 | −84% | 10.8 | 57% |
| | 2 | 1.8 | 24.5 | 10 | −59% | 15.3 | −38% | 25.8 | 5% |
| | 3 | 1.8 | 42.5 | 19.9 | −53% | 38.2 | −10% | 51.5 | 21% |
| | 4 | 1.8 | 121.8 | 55.6 | −54% | 113.4 | −7% | 125.6 | 3% |
| | 5 | 1.8 | 235.4 | 110.1 | −53% | 216 | −8% | 231.4 | −2% |

TABLE 4
Correlation for the FDG-quantification measures
with the TIA for all phantom studies.

| | Value | TLG_{42%} | TLG_{2.5} | BSL | TLG_{RC} |
|---|---|---|---|---|---|
| TLG_{2.5} ≦ 50 | Slope | 0.731 | 0.727 | 1.189 | 1.119 |
| | R² | 0.511 | 0.876 | 0.981 | 0.959 |
| | CCC | 0.350 | 0.761 | 0.933 | 0.931 |
| TLG_{2.5} > 50 | Slope | 0.694 | 0.952 | 1.015 | 1.127 |
| | R² | 0.986 | 0.999 | 0.998 | 0.991 |
| | CCC | 0.873 | 0.997 | 0.998 | 0.974 |
| All TLG | Slope | 0.695 | 0.948 | 1.018 | 1.127 |
| | R² | 0.971 | 0.997 | 0.997 | 0.991 |
| | CCC | 0.906 | 0.996 | 0.998 | 0.984 |

TABLE 5
25 patients with lung tumors with TLG_{2.5} ≦50 ml * SUV.

| | CT- | Recovery | | | | | |
|---|---|---|---|---|---|---|---|
| Patients | Volume | Coefficient | SUV [symbol] | TLG_{42%} | TLG_{2.5} | BSL | TLG [symbol] |
| PA39 | 7.1 | 0.79 | 1.5 | 9.9 | 0.0 | 15.2 | 13.3 |
| PA48 | 1.1 | 0.55 | 2.2 | 3.5 | 0.0 | 7.7 | 4.5 |
| PA22 | 0.6 | 0.47 | 2.8 | 6.2 | 0.5 | 3.6 | 3.6 |
| PA44 | 4.6 | 0.73 | 2.8 | 13.2 | 0.8 | 16.8 | 17.5 |
| PA07 | 2.3 | 0.64 | 3.0 | 7.1 | 1.1 | 12.1 | 10.8 |
| PA46 | 1.4 | 0.57 | 4.0 | 6.1 | 3.2 | 17.8 | 9.4 |
| PA13 | 1.2 | 0.56 | 4.6 | 5.3 | 4.2 | 39.1 | 9.9 |
| PA18 | 1.8 | 0.61 | 3.6 | 8.4 | 4.2 | 17.6 | 10.7 |
| PA15 | 4.0 | 0.71 | 4.1 | 15.2 | 5.5 | 31.8 | 22.8 |
| PA32 | 1.9 | 0.62 | 4.2 | 14.7 | 7.1 | 16.2 | 12.9 |
| PA37 | 1.0 | 0.53 | 5.2 | 11.3 | 8.3 | 31.5 | 9.7 |
| PA02 | 1.2 | 0.56 | 6.9 | 7.7 | 8.8 | 13.6 | 14.9 |
| PA12 | 1.2 | 0.56 | 9.9 | 6.1 | 9.3 | 21.2 | 21.4 |
| PA10 | 4.1 | 0.72 | 6.3 | 9.5 | 10.5 | 36.7 | 36.0 |
| PA38 | 1.0 | 0.53 | 11.1 | 7.7 | 12.5 | 33.8 | 20.2 |
| PA11 | 2.4 | 0.65 | 8.4 | 11.2 | 14.8 | 35.9 | 31.0 |
| PA09 | 2.8 | 0.67 | 9.2 | 13.2 | 18.1 | 20.0 | 38.7 |
| PA29 | 1.7 | 0.60 | 7.0 | 14.9 | 18.2 | 51.6 | 19.8 |
| PA47 | 2.4 | 0.65 | 10.6 | 12.0 | 19.2 | 54.6 | 39.3 |
| PA24 | 5.1 | 0.74 | 8.3 | 17.3 | 26.1 | 2.0 | 56.7 |
| PA34 | 3.0 | 0.68 | 11.8 | 15.3 | 26.7 | 52.0 | 52.2 |
| PA16 | 1.9 | 0.62 | 13.0 | 18.2 | 34.9 | 47.7 | 39.9 |
| PA21 | 2.5 | 0.65 | 14.5 | 19.7 | 35.5 | 99.5 | 55.4 |
| PA17 | 4.9 | 0.74 | 7.5 | 30.7 | 37.1 | 49.0 | 49.7 |
| PA27 | 6.0 | 0.76 | 10.0 | 19.8 | 50.0 | 80.6 | 78.2 |

[symbol]

indicates data missing or illegible when filed

TABLE 6
25 patients with lung tumors with TLG_{2.5} >50 ml * SUV.

| | CT- | Recovery | | | | | |
|---|---|---|---|---|---|---|---|
| | Vol | Co- | | | | | |
| Patients | ume | efficient | SUV [symbol] | TLG_{42%} | TLG_{2.5} | BSL | TLG [symbol] |
| PA06 | 11.2 | 0.85 | 14.3 | 24.2 | 51.5 | 91.3 | 189.0 |
| PA03 | 4.8 | 0.74 | 8.7 | 37.9 | 55.7 | 97.2 | 56.5 |
| PA43 | 5.8 | 0.76 | 8.0 | 43.3 | 56.6 | 105.2 | 61.1 |
| PA41 | 7.8 | 0.80 | 13.6 | 35.9 | 60.4 | 102.6 | 132.5 |
| PA04 | 14.1 | 0.87 | 13.7 | 47.9 | 81.4 | 153.1 | 221.2 |
| PA23 | 9.7 | 0.83 | 11.3 | 71.6 | 98.8 | 156.5 | 132.2 |
| PA01 | 22.0 | 0.93 | 12.0 | 63.6 | 106.5 | 160.3 | 282.1 |
| PA45 | 8.6 | 0.81 | 14.6 | 62.1 | 108.3 | 162.3 | 155.1 |
| PA30 | 13.5 | 0.87 | 10.7 | 97.6 | 128.6 | 243.7 | 166.5 |
| PA40 | 23.1 | 0.94 | 10.1 | 120.7 | 163.0 | 226.0 | 249.8 |
| PA05 | 29.0 | 0.97 | 11.4 | 116.5 | 175.5 | 245.4 | 342.4 |
| PA19 | 5.9 | 0.76 | 10.3 | 134.0 | 228.2 | 369.0 | 80.0 |
| PA36 | 14.4 | 0.88 | 24.0 | 149.3 | 265.1 | 312.7 | 394.3 |
| PA14 | 26.4 | 0.96 | 18.7 | 204.2 | 386.8 | 456.2 | 515.4 |
| PA00 | 52.9 | 1.05 | 11.6 | 392.4 | 483.3 | 441.4 | 588.7 |
| PA25 | 30.4 | 0.97 | 23.9 | 302.7 | 554.2 | 560.7 | 745.0 |
| PA42 | 29.2 | 0.97 | 11.4 | 177.9 | 582.1 | 375.5 | 344.2 |
| PA33 | 154.2 | 1.18 | 15.6 | 347.4 | 719.0 | 905.1 | 2030.6 |
| PA35 | 112.1 | 1.14 | 13.5 | 651.1 | 874.9 | 911.5 | 1323.3 |
| PA28 | 83.6 | 1.10 | 15.9 | 793.5 | 1007.2 | 1069.5 | 1207.6 |
| PA31 | 69.0 | 1.08 | 26.9 | 364.9 | 1075.4 | 1189.4 | 1722.8 |
| PA26 | 127.6 | 1.16 | 19.8 | 1142.7 | 1475.9 | 1636.5 | 2178.7 |
| PA20 | 132.1 | 1.16 | 35.7 | 770.6 | 1809.0 | 1982.7 | 4049.4 |
| PA49 | 137.3 | 1.17 | 23.3 | 1317.9 | 1810.4 | 1969.4 | 2742.5 |
| PA08 | 385.0 | 1.30 | 11.7 | 1271.2 | 1942.9 | 2074.5 | 3469.3 |

[symbol]

indicates data missing or illegible when filed

TABLE 7
Correlation for the FDG-quantification measures with
the surrogate reference for the 50 lung lesions:

| | Value | TLG_{42%} | TLG_{2.5} | BSL | TLG_{RC} |
|---|---|---|---|---|---|
| TLG_{2.5} ≦ 50 | Slope | 0.408 | 0.548 | 1.117 | |
| | R² | 0.863 | 0.906 | 0.833 | |
| | CCC | 0.297 | 0.589 | 0.680 | |
| TLG_{2.5} > 50 | Slope | 0.618 | | 1.084 | 1.705 |
| | R² | 0.942 | | 0.993 | 0.947 |
| | CCC | 0.799 | | 0.987 | 0.743 |

### EXEMPLARY REFERENCES

The following references are hereby incorporated by reference in their entirety.
1. Chang K P, Tsang N M, Liao C T, et al. Prognostic significance of 18F-FDG PET parameters and plasma Epstein-Barr virus DNA load in patients with nasopharyngeal carcinoma. Journal of nuclear medicine: official publication, Society of Nuclear Medicine. January 2012; 53(1):21-28.
2. Groves A M, Shastry M, Rodriguez-Justo M, et al. (1)(8)F-FDG PET and biomarkers for tumour angiogenesis in early breast cancer. European journal of nuclear medicine and molecular imaging. January 2011; 38(1):46-52.
3. Weber W A, Figlin R. Monitoring cancer treatment with PET/CT: Does it make a difference? Journal of Nuclear Medicine. January 2007; 48:36s-44s.
4. Wahl R L, Jacene H, Kasamon Y, Lodge M A. From RECIST to PERCIST: Evolving Considerations for PET response criteria in solid tumors. J Nucl Med. May 2009; 50 Suppl 1:122S-150S.
5. Boellaard R, O'Doherty M J, Weber W A, et al. FDG PET and PET/CT: EANM procedure guidelines for tumour PET imaging: version 1.0. Eur J Nucl Med Mol Imaging. January 2010; 37(1):181-200.
6. Burger I A, Huser D M, von Schulthess G K, Trinckauf J, Burger C, Buck A. Repeatability of FDG quantification in tumor imaging: averaged SUVs are superior to SUVmax. Nuclear medicine and biology. 2012; In press.
7. de Langen A J, Vincent A, Velasquez L M, et al. Repeatability of 18F-FDG uptake measurements in tumors: a metaanalysis. J Nucl Med. May 2012; 53(5):701-708.
8. Schwartz J, Humm J L, Gonen M, et al. Repeatability of SUV measurements in serial PET. Medical physics. May 2011; 38(5):2629-2638.
9. Larson S M, Erdi Y, Akhurst T, et al. Tumor Treatment Response Based on Visual and Quantitative Changes in Global Tumor Glycolysis Using PET-FDG Imaging. The Visual Response Score and the Change in TLGTota1 Lesion Glycolysis. Clinical positron imaging: official journal of the Institute for Clinical P.E.T. May 1999; 2(3):159-171.
10. Seol Y M, Kwon B R, Song M K, et al. Measurement of tumor volume by PET to evaluate prognosis in patients with head and neck cancer treated by chemo-radiation therapy. Acta oncologica. 2010; 49(2):201-208.
11. Chung H H, Kim J W, Han K H, et al. Prognostic value of metabolic tumor volume measured by FDG-PET/C T in patients with cervical cancer. Gynecol Oncol. February 2011; 120(2):270-274.
12. Chung H H, Kwon H W, Kang K W, et al. Prognostic value of preoperative metabolic tumor volume and total lesion glycolysis in patients with epithelial ovarian cancer. Ann Surg Oncol. June 2012; 19(6):1966-1972.
13. Lim R, Eaton A, Lee N Y, et al. 18F-FDG PET/CT Metabolic Tumor Volume and Total Lesion Glycolysis Predict Outcome in Oropharyngeal Squamous Cell Carcinoma. J Nucl Med. October 2012; 53(10):1506-1513.
14. Liu F Y, Chao A, Lai C H, Chou H H, Yen T C. Metabolic tumor volume by 18F-FDG PET/C T can be prognostic for stage IVB endometrial carcinoma. Gynecol Oncol. June 2012; 125(3):566-571.
15. Liao S, Penney B C, Wroblewski K, et al. Prognostic value of metabolic tumor burden on 18F-FDG PET in nonsurgical patients with non-small cell lung cancer. Eur J Nucl Med Mol Imaging. January 2012; 39(1):27-38.
16. Chen H H, Chiu N T, Su W C, Guo H R, Lee B F. Prognostic value of whole-body total lesion glycolysis at pretreatment FDG PET/CT in non-small cell lung cancer. Radiology. August 2012; 264(2):559-566.
17. Hyun S H, Choi J Y, Shim Y M, et al. Prognostic value of metabolic tumor volume measured by 18F-fluorodeoxyglucose positron emission tomography in patients with esophageal carcinoma. Ann Surg Oncol. January 2010; 17(1):115-122.
18. Lee S J, Choi J Y, Lee H J, et al. Prognostic Value of Volume-Based (18)F-Fluorodeoxyglucose PET/CT Parameters in Patients with Clinically Node-Negative Oral Tongue Squamous Cell Carcinoma. Korean J Radiol. November 2012; 13(6):752-759.
19. Abd El-Hafez Y G, Moustafa H M, Khalil H F, Liao C T, Yen T C. Total lesion glycolysis: A possible new prognostic parameter in oral cavity squamous cell carcinoma. Oral Oncol. Oct. 1 2012.
20. Kim T M, Paeng J C, Chun I K, et al. Total lesion glycolysis in positron emission tomography can be a better predictor of outcome than the International Prognostic Index for patients with diffuse large B cell lymphoma. Cancer. Dec. 4 2012.
21. Tylski P, Stute S, Grotus N, et al. Comparative assessment of methods for estimating tumor volume and standardized uptake value in (18)F-FDG PET. Journal of nuclear medicine: official publication, Society of Nuclear Medicine. February 2010; 51(2):268-276.
22. Nestle U, Kremp S, Schaefer-Schuler A, et al. Comparison of different methods for delineation of 18F-FDG PET-positive tissue for target volume definition in radiotherapy of patients with non-Small cell lung cancer. J Nucl Med. August 2005; 46(8):1342-1348.
23. Biehl K J, Kong F M, Dehdashti F, et al. 18F-FDG PET definition of gross tumor volume for radiotherapy of non-small cell lung cancer: can be a single standardized uptake value threshold approach appropriate? Journal of nuclear medicine: official publication, Society of Nuclear Medicine. November 2006; 47(11):1808-1812.
24. Freedman D, Diaconis P. On the Histogram as a Density Estimator—L2 Theory. Z Wahrscheinlichkeit. 1981; 57(4):453-476.
25. Lin L I. A Concordance Correlation-Coefficient to Evaluate Reproducibility. Biometrics. March 1989; 45(1):255-268.
26. Aristophanous M, Penney B C, Martel M K, Pelizzari C A. A Gaussian mixture model for definition of lung tumor volumes in positron emission tomography. Med Phys. November 2007; 34(11):4223-4235.
27. Fox J J, Autran-Blanc E, Morris M J, et al. Practical approach for comparative analysis of multilesion molecular imaging using a semiautomated program for PET/CT. J Nucl Med. November 2011; 52(11):1727-1732.
28. Nehmeh S A, El-Zeftawy H, Greco C, et al. An iterative technique to segment PET lesions using a Monte Carlo based mathematical model. Med Phys. October 2009; 36(10):4803-4809.
29. Hoffman E J, Huang S C, Phelps M E. Quantitation in positron emission computed tomography: 1. Effect of object size. J Comput Assist Tomogr. June 1979; 3(3):299-308.
30. Soret M, Bacharach S L, Buvat I. Partial-volume effect in PET tumor imaging. J Nucl Med. June 2007; 48(6):932-945.

What is claimed is:

1. A non-transitory computer-accessible medium having stored thereon computer-executable instructions for determining at least one characteristic of at least one tissue, wherein, when a computer arrangement executes the instructions, the computer arrangement is configured to perform procedures comprising:
receiving first information related to a volume of interest (VOI) of the at least one tissue;
receiving second information related to a histogram based on the at least one tissue;
determining a Gaussian fit of the histogram; and
determining the at least one characteristic of the at least one tissue by at least partially subtracting or removing the Gaussian fit from the VOI.

2. The computer-accessible medium of claim 1, wherein the Gaussian fit is over a peak of the histogram.

3. The computer-accessible medium of claim 1, wherein the computer hardware arrangement is further configured to determine the histogram based on voxels of the VOI as a function of a standard uptake value of the at least one tissue.

4. The computer-accessible medium of claim 1, wherein the computer hardware arrangement is further configured to bin the histogram based on a Freedman-Diaconis rule.

5. The computer-accessible medium of claim 4, wherein a width of the bin is ΔSUV_{fd}=2 IQR(VOI) N^{-1/3}, where ΔSUV_{fd} is a [¹⁸F]-Fluorodeoxyglucose standard uptake value, IQR(VOI) is an interquartile range (VOI), and N is a number of voxels in the IQR(VOI).

6. The computer-accessible medium of claim 1, wherein the at least one tissue is a tumor.

7. The computer-accessible medium of claim 6, wherein the at least one characteristic is a tumor activity.

8. The computer-accessible medium of claim 7, wherein the tumor activity is a background subtracted lesion activity (BSL).

9. The computer-accessible medium of claim 1, wherein the at least one characteristic is a tracer uptake metric.

10. The computer-accessible medium of claim 1, wherein the computer hardware arrangement is further configured to determine a mean BSL based on a mode of the histogram.

11. The computer-accessible medium of claim 10, wherein the Gaussian fit is over a region defined by SUV_{BG}±SUV_{V}_{ₘₐₓ}_{/2}, where SUV_{BG} is the mean BSL standard uptake value (SUV) and SUV_{V}_{ₘₐₓ}_{/2} is an SUV of histogram bins located at a half maximum of the mode.

12. The computer-accessible medium of claim 11, wherein the computer hardware arrangement is further configured to (i) set all negative values to zero, and (ii) subtract the Gaussian fit from the histogram based on the zero values.

13. The computer-accessible medium of claim 1, wherein the computer hardware arrangement is further configured to generate the second information using positron emission tomography.

14. The computer-accessible medium of claim 1, wherein the computer hardware arrangement is further configured to generate at least one image of the at least one tissue based on the at least one characteristic.

15. The computer-accessible medium of claim 1, wherein the computer hardware arrangement is further configured to receive a selection of the VOI.

16. The computer-accessible medium of claim 15, wherein the VOI is selected by a user.

17. The computer-accessible medium of claim 1, wherein the computer hardware arrangement is further configured to approximate background data in the histogram as a normal distribution.

18. The computer-accessible medium of claim 1, wherein the VOI is a total VOI.

19. A method for determining at least one characteristic of at least one tissue, comprising:
receiving first information related to a volume of interest (VOI) of the at least one tissue;
receiving second information related to a histogram based on the at least one tissue;
determining a Gaussian fit of the histogram; and
using a computer hardware arrangement, determining the at least one characteristic of the at least one tissue by at least partially subtracting or removing the Gaussian fit from the VOI.

20. A system for determining at least one characteristic of at least one tissue, comprising:
a computer hardware arrangement configured to:
receive first information related to a volume of interest (VOI) of the at least one tissue;
receive second information related to a histogram based on the at least one tissue;
determine a Gaussian fit of the histogram; and
determine the at least one characteristic of the at least one tissue by at least partially subtracting or removing the Gaussian fit from the VOI.
